(19) United States
(12) Patent Application Publication
(10) Pub. No.: US 2008/0139840 A1
(43) Pub. Date: Jun. 12, 2008

(54) PROCESS FOR PREPARING ALKYL ARYL SULPHONIC ACIDS AND ALKYL ARYL SULPHONATES

(76) Inventors: Matthew Thomas Anderson, Ngaio (NZ); Stacy John Archibald, Lower Hutt (NZ); Hendrik Dirkzwager, Amstermam (NL); Wayne Ashley Thomas, Lower Hutt (NZ)

Correspondence Address: Shell Oil Company, 910 Louisiana, Houston, TX, 77002, US

(21) Appl. No.: 11/933,995

(22) Filed: Nov. 1, 2007

(30) Foreign Application Priority Data
Nov. 3, 2006 (EP) 06255684.0

(51) Int. Cl. C07C 309/01

(52) U.S. Cl. 562/123

(57) ABSTRACT

A process for preparing an alkyl aryl sulphonic acid comprising the steps of:
(a) contacting an alkyl aromatic hydrocarbon with a gaseous sulphonating agent to produce (i) a first liquid reaction product comprising an alkyl aryl sulphonic acid and (ii) a gaseous effluent stream;
(b) separating the first liquid reaction product from the gaseous effluent stream;
(c) purifying the gaseous effluent stream to provide a cleaned gaseous stream and a second liquid reaction product;
(d) recycling the second liquid reaction product to the first liquid reaction product produced after separation step (b) to produce a third liquid reaction product comprising alkyl aryl sulphonic acid;
   wherein the alkyl aromatic hydrocarbon is obtained by contacting an aromatic hydrocarbon with an olefin under alkylating conditions, and wherein said olefin is obtained by dehydrogenation of a Fischer-Tropsch derived paraffinic feedstock.

### FIELD OF THE INVENTION

This invention relates to a process for preparing alkyl aryl sulphonic acids and alkyl aryl sulphonates.

### BACKGROUND OF THE INVENTION

Alkyl aryl sulphonates are important compounds for use as surfactants in detergent compositions. They are produced commercially by sulphonation of alkyl aryl hydrocarbons. The main sulphonation reactions in the case of sulfur trioxide as sulphonating agent and alkyl benzene as the alkyl aryl hydrocarbon can be written as follows:

RC₆H₅+2SO₃→RC₆H₄SO₂OSO₃H(pyrosulphonic acid)

RC₆H₄SO₂OSO₃H+RC₆H₅→2RC₆H₄SO₃H(alkyl benzene sulphonic acid)

Typically after stabilisation and hydrolysis treatment, alkyl benzene sulphonic acids are stable compounds which can be stored and transported as such. Alternatively, alkyl benzene sulphonic acids can be neutralized, for example by reaction with a base, to produce alkyl aryl sulphonates in salt form.

Since alkyl aryl sulphonates are frequently used as surfactants in detergent compositions, especially laundry detergent formulations, it is important that they have good detergency, solubility and biodegradability properties. Such properties are influenced by a variety of factors including the type of olefin (e.g. linear or branched) used to alkylate the aryl hydrocarbon and the catalyst used in the alkylation reaction.

The properties of the alkyl aryl sulphonates can also be influenced by the source of the olefin used to alkylate the aryl hydrocarbon. Said olefin can be produced in a variety of ways including oligomerization of ethylene, dehydrogenation of paraffins, and the like. However, in the majority of linear alkyl benzene production plants, the olefin is derived from the dehydrogenation of a paraffinic feedstock. In particular, the paraffinic feedstock is commonly derived from the separation of nonbranched (linear) hydrocarbons or lightly branched hydrocarbons from a kerosene boiling range petroleum fraction. Several known processes that accomplish such a separation are known including the commercial UOP Molex® process. However, in recent years, attention has been focused on making use of cleaner, more cost-effective feedstocks, such as paraffins derived from a Fischer-Tropsch synthesis. Paraffins obtained in a Fischer-Tropsch synthesis are particularly advantageous from an environmental point of view because Fischer-Tropsch products are generally very low in their content of sulphur, nitrogen, oxygenates and cyclic products. Further, Fischer-Tropsch products are cost effective. Other benefits (e.g. detergency benefits in the final alkyl aryl sulphonate product) may be realized from using Fischer-Tropsch derived paraffins, in particular due to the slightly higher branching levels found in Fischer-Tropsch derived paraffins compared to kerosene-derived paraffins.

In conventional sulphonation processes using SO₃ as a sulphonating agent, it is known that a small amount of alkyl aryl sulphonic acid product (in the form of mist droplets) ends up in the exhaust gas exiting the sulphonation reactor after separation from the reactor product. This entrained sulphonic acid product is present in admixture with side products such as sulphuric acid. For environmental reasons, it is necessary to purify the exhaust gas before it is released into the atmosphere. Purification of the exhaust gas is commonly carried out by passing the exhaust gas through an electrostatic precipitator (ESP) in order to remove the entrained sulphonic acids and sulphuric acid, optionally followed by caustic treatment. Instead of simply discarding the entrained acids, it is desirable, for environmental and process efficiency reasons, to recover and recycle these products. Recycling of the ESP residue is known from “Sulfonation Technology in the Detergent Industry”, W. Herman de Groot, Kluwer Academic Publishers, 1991. However, it is notable that the de Groot reference teaches that recycling of ESP residues is not suitable during the sulphonation or sulphation of all materials. For example, it is mentioned on page 210 of that reference that recycle of ESP residues is not suitable during sulphonation of alpha olefins, or the sulphation of alcohols and alcohol ethoxylates.

While alkyl aryl sulphonates have been produced commercially for many years using conventional sulphonation processes there still exists a need for providing improvements to the process of manufacture, in particular in terms of improving the efficiency and environmental impact of the process. However it is also important that any process improvements do not result in a deterioration in the quality of the final alkyl aryl sulphonate product. In particular, any process improvements should not detrimentally effect product characteristics, such as the colour of the final alkyl aryl sulphonates, to a significant degree.

The benefits of ESP recycle in the manufacture of alkyl aryl sulphonates has been mentioned above. Separately, the benefits of using Fischer-Tropsch derived paraffins in the manufacture of alkyl aryl sulphonates has been highlighted above, including the detergency benefits that may result from the slightly more branched nature of Fischer-Tropsch derived paraffins compared to conventional kerosene-derived paraffins. Therefore, it would be desirable to use ESP recycle in the manufacture of alkyl aryl sulphonates, wherein the alkyl group has been derived from Fischer-Tropsch paraffins. However, knowing that recycling of ESP residues cannot be applied to all materials (see de Groot reference mentioned above), it would not have been obvious to a person skilled in the art, that recycle of ESP residues could be applied to the sulphonation of an alkyl aryl hydrocarbon wherein the alkyl group is derived from Fischer-Tropsch paraffins (having slightly higher levels of branching) instead of conventional kerosene-based paraffins, without effecting product quality.

### SUMMARY OF THE INVENTION

It has now been found by the present inventors that the process described hereinbelow, which involves recycling of the ESP residues during the sulphonation process for preparing alkyl aryl sulphonates together with the use of Fischer-Tropsch derived feedstocks for preparing the alkyl aryl hydrocarbons, provides a more efficient and more environmentally friendly method for producing alkyl aryl sulphonic acids, while, surprisingly, not detrimentally affecting the properties, in particular, the colour, of the final alkyl aryl sulphonates to a significant degree.

According to one aspect of the present invention there is provided a process for preparing an alkyl aryl sulphonic acid comprising the steps of:
(a) contacting an alkyl aromatic hydrocarbon with a gaseous sulphonating agent to produce (i) a first liquid reaction product (comprising an alkyl aryl sulphonic acid) and (ii) a gaseous effluent stream;
(b) separating the first liquid reaction product from the gaseous effluent stream;
(c) purifying the gaseous effluent stream to provide a cleaned gaseous stream and a second liquid reaction product;
(d) recycling the second liquid reaction product to the first liquid reaction product produced after separation step (b) to produce a third liquid reaction product comprising alkyl aryl sulphonic acid;
   wherein the alkyl aromatic hydrocarbon is obtained by contacting an aromatic hydrocarbon with an olefin under alkylating conditions, and wherein said olefin is obtained by dehydrogenation of a Fischer-Tropsch derived paraffinic feedstock.

According to a second aspect of the present invention there is provided a process for preparing an alkyl aryl sulphonic acid comprising the steps of:
(a1) contacting an aromatic hydrocarbon with an olefin under alkylating conditions in the presence of an alkylation catalyst to produce an alkyl aromatic hydrocarbon, wherein said olefin is obtained by dehydrogenation of a Fischer-Tropsch derived paraffinic feedstock;
(a) contacting the alkyl aromatic hydrocarbon with a gaseous sulphonating agent to produce (i) a first liquid reaction product (comprising alkyl aryl sulphonic acid) and (ii) a gaseous effluent stream;
(b) separating the first liquid reaction product from the gaseous effluent stream;
(c) purifying the gaseous effluent stream to provide a cleaned gaseous stream and a second liquid reaction product;
(d) recycling the second liquid reaction product to the first liquid reaction product produced after separation step (b) to produce a third liquid reaction product comprising alkyl aryl sulphonic acid.

### BRIEF DESCRIPTION OF THE DRAWINGS

FIG. 1 is a block flow diagram of the process according to the first aspect of the present invention.

FIG. 2 is a block flow diagram of the process according to the second aspect of the present invention.

### DETAILED DESCRIPTION OF THE INVENTION

An important step of the process herein involves the sulphonation of an alkyl aromatic hydrocarbon in which an alkyl aromatic hydrocarbon is contacted with a gaseous sulphonating agent.

In the process of the present invention, alkyl aromatic hydrocarbons may be sulphonated by any method of sulphonation which is known in the art which uses a gaseous sulphonating agent.

A preferred sulphonating agent for use herein is sulphur trioxide. A commonly employed method of using sulphur trioxide is as a vapour diluted with an inert dry carrier gas, usually air, to give a dilute sulphur trioxide gas stream preferably containing from about 2 to about 20 volume percent sulphur trioxide. Details of a preferred sulphonation method, which involves using an air/sulphur trioxide mixture, are known from U.S. Pat. No. 3,427,342, which is herein incorporated by reference in its entirety.

Sulphonation conditions will depend on the sulphonating agent used but are well known to those skilled in the art. Sulphonation with sulphur trioxide is most often performed in the temperature range of from about 25° C. to about 120° C., although more usually the reaction temperature is kept under 100° C. and a preferred temperature range is in the range of from 30° C. to about 75° C. Typical reaction pressures for sulphonation with sulphur trioxide are pressures up to about 50 kPa above atmospheric pressure, preferably in the range of from about 30 kPa to about 50 kPa above atmospheric pressure. Typically the ratio of sulphur trioxide to alkyl aromatic hydrocarbon is in the range of from about 1.05:1 to about 1.2:1.

A relatively large number of processes have been developed for sulphonation of detergent alkylates. For example, U.S. Pat. No. 3,169,142 uses a flowing film of the detergent alkylate with a pressurized stream of an inert diluent and a vaporized sulphur trioxide, where the inert diluent may be dry air, nitrogen, carbon dioxide, carbon monoxide, sulfur dioxide, a halogenated hydrocarbon, or a low molecular weight paraffinic hydrocarbon such as methane, ethane, propane, butane, or a mixture thereof. Sulphur trioxide is diluted with a gas within the range of 5:1 to 50:1 by volume. U.S. Pat. No. 3,328,460 describes sulphonation using a gas mixture of inert gas and gaseous sulphur trioxide where the detergent alkylate is reacted as a liquid film on the order of 0.002-0.003 inch thick at a reaction temperature of about 30° C. U.S. Pat. No. 3,535,339 uses gaseous sulphur trioxide at subatmospheric pressure without a gaseous diluent and also uses a thin flowing film of liquid detergent alkylate for reaction. At another extreme is U.S. Pat. No. 3,198,849 which describes an exothermic sulphonation between an alkylbenzene and undiluted gaseous sulphur trioxide. U.S. Pat. No. 3,427,342 describes the sulphonation of alkylbenzenes using gaseous sulphur trioxide in a mole ratio of 1.05:1 to about 1.15:1. In that patent the sulphur trioxide is controlled at 2-8% by volume and most preferably an 8-10 mole percent excess of sulphur trioxide is used relative to the alkylbenzene. Although the average temperature in the reaction mixture zone is 30-55° C., the temperature in the reaction zone, which is only a short portion of the reaction mixture zone, is substantially higher at 66-93° C. U.S. Pat. No. 3,169,142, U.S. Pat. No. 3,328,460, U.S. Pat. No. 3,535,339, U.S. Pat. No. 3,198,849 and U.S. Pat. No. 3,427,342 are herein incorporated by reference in their entirety.

Additional references to sulphonation, including details of sulphonation reactors, sulphonation chemistry, sulphonation process conditions, and the like, may be found in “Sulphonation Technology in the Detergent Industry”, W. Herman de Groot, Kluwer Academic Publishers, 1991.

In the processes of the present invention, reaction of the alkyl aromatic hydrocarbon with a sulphonating agent produces (i) a first liquid reaction product (comprising an alkyl aryl sulphonic acid) and (ii) a gaseous effluent stream.

Typically the gaseous effluent stream emerging from the sulphonation reactor comprises sulphur oxides (typically unconverted SO₂ and unreacted SO₃), sulphuric acid (in the form of mist) and entrained alkyl aryl sulphonic acid (in the form of mist droplets).

After sulphonation step (a) the first liquid reaction product is separated from the gaseous effluent stream (separation step (b)). This separation step is carried out using any known method for separating gases and liquids, including, for example, distillation, heating and by means of a Gas-Liquid Separator. A preferred method herein for separating the first liquid reaction product from the gaseous effluent stream is by means of a Gas-Liquid Separator. Typically this consists of a vessel with a tangential side inlet. The liquid leaves the vessel as a bottom stream and the gas leaves the vessel via an outlet at the top of the vessel.

As mentioned above, the gaseous effluent stream emerging from the sulphonation reactor typically comprises sulphur oxides, sulphuric acid (in the form of mist) and entrained alkyl aryl sulphonic acid (in the form of mist droplets). Therefore, after separation of the first liquid reaction product from the gaseous effluent stream, the gaseous effluent stream must be purified before emission to ambient atmosphere. In the process of the present invention, the effluent gaseous stream is purified to provide a cleaned gaseous stream and a second liquid reaction product. The purification step can be carried out using any purification technique known in the art, including, centrifugation, absorption, electrostatic precipitation, and the like. A preferred method of purification for use herein is by means of an electrostatic precipitator (ESP) to trap sulphuric acid mist and entrained alkyl aryl sulphonic acid mist. Hence the second liquid reaction product emerging from the purification step typically comprises sulphuric acid and alkyl aryl sulphonic acid.

An important step in the process involves recycling of the second liquid reaction product to the first liquid reaction product produced after separation step (b) to produce a third liquid reaction product comprising alkyl aryl sulphonic acid. It is found that such a recycling step does not detrimentally effect the colour of the final linear alkyl benzene sulphonate product to any significant degree, despite the presence of undesirable impurities, such as, for example, sulphuric acid, in the second liquid reaction product emerging from the purification step described above.

Preferably after stabilization and hydrolysis, the final alkyl benzene sulphonic acid is a stable product which can be stored and transported as such. However, in order to convert the alkyl aryl sulphonic acid in the third liquid reaction product to an alkyl aryl sulphonate, the sulphonic acid may be subjected to a neutralization step. Said neutralization step is carried out using any suitable neutralization agent known to those skilled in the art, for example, by neutralization of the alkyl aryl sulphonic acid with a base to form the alkyl arylsulphonate in the form of a salt. Suitable bases are the hydroxides of alkali metals and alkaline earth metals; and ammonium hydroxides, which provide the cation M of the salts as specified below.

Further, optional, reaction steps may be required before sulphonic acid neutralization. These optional reaction steps will be well known to those skilled in the art of sulphonation. For example, alkyl benzene sulphonic acid typically passes to an ageing step for conversion of any intermediate products (such as pyrosulphonic acid) to the desired alkyl benzene sulphonic acid. In addition, a hydrolysis or stabilization step is usually required to convert certain intermediates, like alkyl benzene sulphonic acid anhydrides, to alkyl benzene sulphonic acid with a small amount of water (approximately 1% on alkyl benzene sulphonic acid).

Other further, optional, steps may also be carried out. Such optional steps will be well known to those skilled in the art of sulphonation. For example, the cleaned gaseous stream exiting the purification step described above, e.g. exiting the Electrostatic Precipitator, may be subjected to a caustic scrubbing step (by contacting the cleaned gaseous stream with caustic soda) before being released into the environment, in order to remove small amounts of SO₂ and gaseous SO₃ that pass the purification step.

The general class of alkyl arylsulphonates which may be made in accordance with this invention can be characterized by the chemical formula (R-A′-SO₃)ₙM, wherein R represents an alkyl group having a carbon number in the range of from 7 to 35, in particular from 7 to 18, more in particular from 10 to 18, most in particular from 10 to 13; A′ represents a divalent aromatic hydrocarbyl group, in particular a phenylene group; M is a cation selected from an alkali metal ion, an alkaline earth metal ion, an ammonium ion, and mixtures thereof; and n is a number depending on the valency of the cation(s) M, such that the total electrical charge is zero. The ammonium ion may be derived from an organic amine having 1, 2 or 3 organic groups attached to the nitrogen atom. Suitable ammonium ions are derived from monoethanol amine, diethanol amine and triethanol amine. It is preferred that the ammonium ion is of the formula NH₄⁺. In preferred embodiments M represents sodium, potassium or magnesium. Potassium ions can promote the water solubility of the alkyl arylsulphonates and magnesium can promote their performance in soft water.

The alkyl aromatic hydrocarbon used herein is prepared by contacting an olefin with an aromatic compound under suitable alkylation conditions. This can be performed under a large variety of alkylating conditions. Preferably, the said alkylation leads to monoalkylation, and only to a lesser degree to dialkylation or higher alkylation, if any.

The aromatic hydrocarbon applicable in the alkylation may be one or more of benzene, toluene, xylene, for example o-xylene or a mixture of xylenes; and naphthalene. Preferably the aromatic hydrocarbon is benzene.

The olefin used in the alkylation process is obtained by dehydrogenation of a Fischer-Tropsch derived paraffinic feedstock. Fischer-Tropsch derived paraffinic feedstocks are useful herein in combination with the recycle of ESP residues in order to provide an improved process for the manufacture of alkyl aryl sulphonates in terms of improving the efficiency and environmental impact of the process. It is particularly surprising that the combination of these two features does not have a significantly detrimental effect on the properties of the final alkyl aryl sulphonate product.

Paraffins obtained in a Fischer Tropsch synthesis are particularly advantageous for use herein because Fischer Tropsch products are generally very low in their content of sulphur, nitrogen, oxygenates and cyclic products and they are cost effective.

The paraffinic feedstock preferably comprises nonbranched (linear) or normal paraffin molecules having a total number of carbon atoms per paraffin molecule of generally from about 7 to about 35, preferably from about 7 to about 18, more preferably from about 10 to about 18, especially from about 10 to about 13 carbon atoms.

In addition to nonbranched paraffins the paraffinic feedstock may also contain other acyclic compounds such as, for example, lightly branched paraffins having one or more alkyl groups branches selected from methyl, ethyl and propyl groups. Preferably the lightly branched paraffins have only one alkyl branch. The paraffinic feedstock is normally a mixture of linear and lightly branched paraffins having different carbon numbers.

The paraffinic feedstock is subjected to a dehydrogenation step in order to convert the paraffins into olefins. The paraffinic feedstock is contacted with a hydrogen stream in the presence of a dehydrogenation catalyst under dehydrogenation reaction conditions. The skilled person is aware of the techniques of preparing the catalysts, performing the dehydrogenation step and performing associated separation steps, for use in this invention. Suitable dehydrogenation catalysts are well known in the art and are exemplified in U.S. Pat. No. 3,274,287, U.S. Pat. No. 3,315,007, U.S. Pat. No. 3,315,008, U.S. Pat. No. 3,745,112, U.S. Pat. No. 4,430,517, U.S. Pat. No. 4,716,143, U.S. Pat. No. 4,762,960, U.S. Pat. No. 4,786,625, U.S. Pat. No. 4,827,072 and U.S. Pat. No. 6,187,981, which are herein incorporated by reference in their entirety. Dehydrogenation conditions include a temperature of from 400° C. to 900° C., preferably from 400° C. to 525° C. and a pressure of from 1 kPa to about 1013 kPa and a LHSV (linear hour space velocity) of 0.1 to 100 hour⁻¹.

A preferred dehydrogenation process for use herein is the PACOL® process from UOP which uses a platinum-based dehydrogenation catalyst. Diolefins present after the dehydrogenation reaction can be converted to monolefins using the DEFINE® process from UOP.

The olefin feedstock used in the alkylation step may comprise paraffins which were not converted in the dehydrogenation step. Such non-converted paraffins may suitably be removed in a subsequent stage, in particular during the work-up of the alkylation reaction mixture, as described hereinafter, and recycled to the dehydrogenation step. Typically, the quantity of the olefinic portion present in such an olefin/paraffin mixture is in the range of from 1 to 50% mole relative to the total number of moles of olefins and paraffins present, more typically in the range of from 5 to 30% mole, in particular from 10 to 20% mole, on the same basis. Typically quantity of the paraffinic portion present in such an olefin/paraffin mixture is in the range of from 50 to 99% mole relative to the total number of moles of olefins and paraffins present, more typically in the range of from 70 to 95% mole, in particular from 80 to 90% mole, on the same basis.

The molar ratio of the aromatic hydrocarbons to the olefins may be selected from a wide range. In order to favor monoalkylation, this molar ratio is suitably at least 1, in particular at least 7.

The catalyst used for the alkylation process can be any catalyst suitable for use as an alkylation catalyst. Typical catalysts for alkylation include homogeneous Lewis acids including metal halides such as aluminium trichloride, Bronsted acids such as hydrogen fluoride, sulphuric acid, and phosphoric acid, and heterogeneous catalysts such as amorphous and crystalline silica alumina. Narrow pore zeolites, such as dealuminated mordenite, offretite and Beta zeolite, give higher selectivity to alkylation towards the end positions of the alkyl chain, typically on the 2-position of the alkyl chain.

The said alkylation may or may not be carried out in the presence of a liquid diluent. Suitable diluents are, for example, paraffin mixtures of a suitable boiling range, such as the paraffins which were not converted in the dehydrogenation and which were not removed from the dehydrogenation product. An excess of the aromatic hydrocarbon may act as a diluent.

The preparation of alkyl aromatic hydrocarbons by contacting an olefin with an aromatic hydrocarbon may be performed under alkylating conditions involving reaction temperatures selected from a large range. The reaction temperature is suitably selected in the range of from 30° C. to 300° C., however the reaction temperature is dependent on the type of alkylation process and catalyst used.

The general class of alkyl aromatic compounds which may be made herein can be characterised by the chemical formula R-A, wherein R represents an alkyl group derived from the olefins according to this invention by the addition thereto of a hydrogen atom, which olefins have a carbon number in the range of from 7 to 35, in particular from 7 to 18, more in particular from 10 to 18, most in particular from 10 to 13; and A represents an aromatic hydrocarbyl group, in particular a phenyl group.

The alkyl arylsulphonate surfactants prepared in accordance with this invention may be used as surfactants in a wide variety of applications, including detergent formulations such as granular laundry detergent formulations, liquid laundry detergent formulations, liquid dishwashing detergent formulations; and in miscellaneous formulations such as general purpose cleaning agents, liquid soaps, shampoos and liquid scouring agents.

The alkyl arylsulphonate surfactants prepared in accordance with the present invention find particular use in detergent formulations, specifically laundry detergent formulations. These formulations are generally comprised of a number of components, besides the alkyl arylsulphonate surfactants themselves such as other surfactants of the ionic, nonionic, amphoteric or cationic type, builders, cobuilders, bleaching agents and their activators, foam controlling agents, enzymes, anti-greying agents, optical brighteners, and stabilisers. Selection of suitable additional components, including their amounts, is well within the ambit of the person skilled in the art of detergent formulation.

The alkyl arylsulphonate surfactants which can be made in accordance with this invention may also advantageously be used in personal care products, in enhanced oil recovery applications and for the removal of oil spillage off-shore and on inland water-ways, canals and lakes.

The present invention will now be described by way of example with reference to the accompanying drawings.

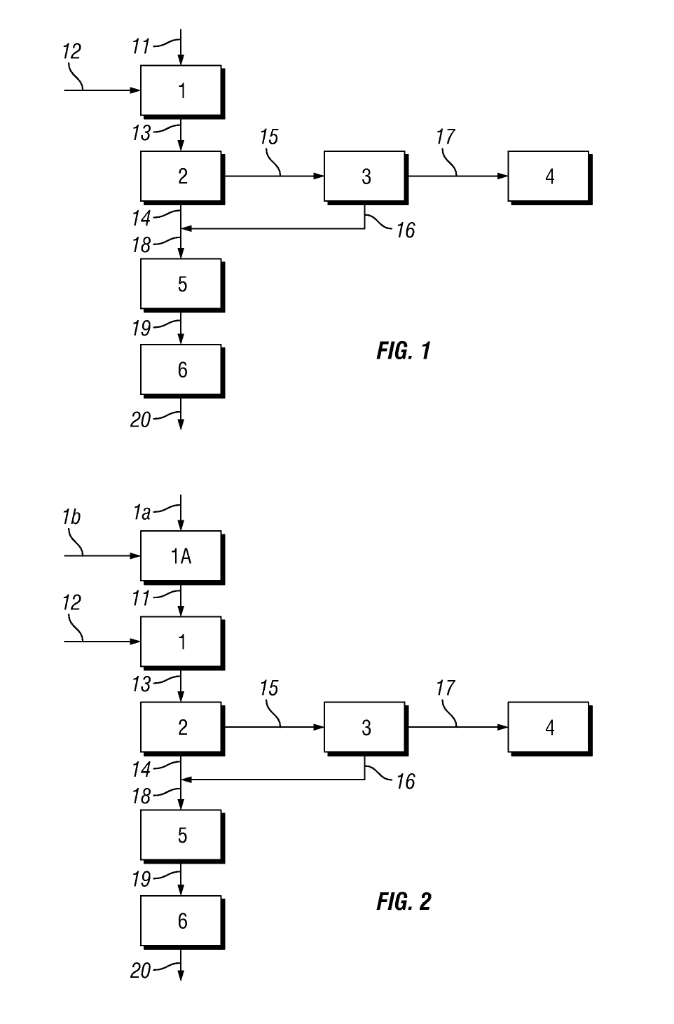

Referring to FIG. 1, Block 1 represents a sulphonation reaction zone. Block 2 represents a gas-liquid separation zone. Block 3 represents an effluent gas purification zone. Block 4 represents an optional NaOH scrubbing zone. Block 5 represents an optional stabilization and hydrolysis zone. Block 6 represents an optional neutralization zone.

Referring to FIG. 1, Line 11 represents the alkyl aryl hydrocarbon starting material wherein the alkyl group has been derived from Fischer-Tropsch paraffinic feedstock. Line 12 represents a sulphonating agent. Line 13 represents the first liquid reaction product and gaseous effluent stream emerging from the sulphonation reaction zone. Line 14 represents the first liquid reaction product emerging from the gas-liquid separation zone. Line 15 represents the gaseous effluent stream emerging from the gas-liquid separation zone. Line 16 represents the second liquid reaction product emerging from the effluent gas purification zone. Line 17 represents the cleaned gaseous stream emerging from the effluent gas purification zone. Line 18 represents the third liquid reaction product which is a combination of the first liquid reaction product and the second liquid reaction product. Line 19 represents the alkyl sulphonic acid emerging from the optional stablisation and hydrolysis zone. Line 20 represents the alkyl aryl sulphonate emerging from the optional neutralization zone.

Referring to FIG. 2, Block 1A represents an alkylation reaction zone. Line 1a represents an aryl hydrocarbon feedstock. Line 1b represents an olefin feedstock which has been prepared by dehydrogenation of a Fischer-Tropsch derived paraffinic feedstock. All other blocks and lines in FIG. 2 are as described above for FIG. 1.

The present invention will now be illustrated by the following Examples, which should not be regarded as limiting the scope of the present invention in any way.

### EXAMPLE 1

A linear alkyl benzene was prepared by dehydrogenation of a Fischer-Tropsch derived paraffinic feedstock using the PACOL® and DEFINE® processes from UOP, followed by alkylation using HF as alkylation catalyst. The Fischer-Tropsch paraffins were prepared in a Fischer-Tropsch reaction using a cobalt-titania Fischer-Tropsch catalyst. The required carbon fraction is obtained by a combination of distillation and hydrogenation. The resulting Fischer-Tropsch paraffins had the following composition:

| Paraffin Carbon Number | Weight % |
|---|---|
| C9 and lighter | 0.0 |
| C10 | 10.3 |
| C11 | 31.0 |
| C12 | 29.9 |
| C13 | 28.2 |
| C14 and heavier | 0.6 |

The linear alkyl benzene (LAB) was then subjected to a sulphonation reaction by reaction with sulphur trioxide. The sulphur trioxide was prepared using elemental sulphur as base material which was melted, burned to SO₂ and subsequently converted to SO₃. A 6 mol % SO₃/air mixture was fed to a sulphonation reactor at a flow rate of 186 kg sulphur/hour. The sulphonation reactor was a 37 tube Ballestra type F thin film reactor operating at a LAB feed rate of 1250 kg/hour. The sulphonation reaction was carried out a temperature of 50° C. and at a pressure of approximately 30 kPa above atmospheric pressure. Linear alkyl benzene sulphonic acid product stream was separated from the depleted SO₃/air vapour stream in a gas/liquid separator and subsequently routed to an ageing section (2 vessels in series) and thereafter to a hydrolysis vessel where approximately 1% water was added to stabilize the product further. Total residence time of ageing and hydrolysis vessels was approximately 40 minutes and temperature of ageing/hydrolysis section was maintained at 45-50° C.

The depleted SO₃/vapour stream emerging from the gas/liquid separator was then routed to an Electrostatic Precipitator unit (ESP) where traces of liquid (comprising linear alkyl benzene sulphonic acid and sulphuric acid) were removed. The removed acidic liquid was then recycled to the liquid linear alkyl benzene sulphonic acid stream leaving the gas/liquid separator at a rate of 3.5 kg/hour (i.e. before entering the ageing/hydrolysis section). Finally the last traces of acid/SO₃ were removed from the air vapour stream by caustic scrubbing.

The alkyl group of the resulting alkyl aryl sulphonic acid had the following carbon number distribution:

| Alkyl Carbon Number | Weight % |
|---|---|
| Less than C10 | 0.71 |
| C10 | 11.79 |
| C11 | 33.96 |
| C12 | 30.02 |
| C13 | 23.97 |
| C14 and above | 0.26 |

The Absorbance, direct acidity, UOM (unreacted organic matter), water content and sulphuric acid content of samples of the final linear alkyl benzene sulphonic acid product were measured using the various test methods described below. Results are shown in Table 1 below.

### Absorbance Test Method

The absorbance of a 50 g/L solution in ethanol was measured in a 4 cm cell at a wavelength of 400 nm using a single beam UV spectrophotometer. Absorbance measurements are a criteria for color formation. In general, the higher the Absorbance value the more colored the product is.

### Direct Acidity Test Method

Around 1 g of linear alkyl benzene sulphonic acid was accurately weighed and dissolved in 30 mL of EtOH and 30 mL of H₂O and titrated to the equivalence-point with 0.5 mol/L NaOH (expressed as mgKOH/g).

### UOM (Unreacted Organic Matter) Test Method

The UOM of a 50 g/L sample of linear alkyl benzene sulphonic acid in EtOH was measured against a standard of 0.65 g/L linear alkyl benzene in EtOH using HPLC. An ion exchange column was used with a mobile phase of EtOH.

### Test Method to Determine Amount of Water in Linear Alkyl Benzene Sulphonic Acid Sample

The amount of water in a linear alkyl benzene sulphonic acid sample was measured using one component, volumetric Karl-Fischer titration. Sample size was approximately 3.5 g. The titrant efficiency was ≧5.0 mgH₂O/mL and the Karl-Fischer solvent was buffered with 50 g/L imidazole.

### Test Method to Determine Amount of Sulphuric Acid in Linear Alkyl Benzene Sulphonic Acid Sample

The amount of sulphuric acid in a linear alkyl benzene sulphonic acid sample was measured using electrochemical titration using lead nitrate.

### EXAMPLE 2

### Comparative

Example 1 was repeated except that the acidic liquid emerging from the Electrostatic Precipitator (ESP) was not recycled. The Absorbance, direct acidity, UOM (unreacted organic matter), water content and sulphuric acid content of samples of the final linear alkyl benzene sulphonic acid product was measured using the Test Methods described above. Results are shown in Table 1 below.

### EXAMPLE 3

### Comparative

Example 1 was repeated except that the linear alkyl benzene was prepared by dehydrogenation of a C9-C14 kerosene-derived paraffinic feedstock. The alkyl group of the resulting alkyl aryl sulphonic acid has the following carbon number distribution:

| Alkyl carbon number | Weight % |
|---|---|
| C9 + lighter | 0.41 |
| C10 | 10.26 |
| C11 | 34.44 |
| C12 | 33.17 |
| C13 | 21.41 |
| C14 | 0.31 |
| C15 + heavier | Trace |

The Absorbance, direct acidity, UOM (unreacted organic matter), water content and sulphuric acid content of samples of the final linear alkyl benzene sulphonic acid product was measured using the Test Methods described above. Results are shown in Table 1 below.

### EXAMPLE 4

### Comparative

Example 3 was repeated except that the acidic liquid emerging from the Electrostatic Precipitator was not recycled. The Absorbance, direct acidity, UOM (unreacted organic matter), water content and sulphuric acid content of samples of the final linear alkyl benzene sulphonic acid product was measured using the Test Methods described above. Results are shown in Table 1 below.

TABLE 1
Direct Acidity, Absorbance, UOM, Water content and
sulphuric acid content of linear alkyl benzene sulphonic
acid samples prepared in Examples 1 to 4

| | Direct | | | | |
|---|---|---|---|---|---|
| | Acidity mg | | | | H₂SO₄ |
| E.g. | KOH/gr | Absorbance | UOM % w | Water % w | % w |
| 1 | 188.0-191.0 | 0.087-0.094 | 1.12-1.24 | 0.38-0.44 | 1.99 |
| 2 | 186.9-190.1 | 0.054-0.080 | 1.19-1.30 | 0.40-0.46 | 1.84 |
| 3 | 188.1-190.3 | 0.067-0.080 | 1.17-1.23 | 0.39-0.47 | 1.99 |
| 4 | 188.9-192.7 | 0.050-0.074 | 1.15-1.24 | 0.36-0.44 | 1.73 |

Since a number of samples were measured for direct acidity, absorbance, UOM and water content, ranges for these measurements are quoted in Table 1. Only one sample per example was measured for sulphuric acid content hence only one figure is quoted for each example in Table 1 for sulphuric acid content.

It can be seen from Table 1 that the Absorbance of the linear alkyl benzene sulphonic acid produced in Example 1 (using Fischer-Tropsch derived paraffinic feedstock together with recycle of the acidic liquid emerging from the ESP) is not significantly different from the Absorbance of the linear alkyl benzene sulphonic acid produced in Example 2 (without recycle of the acidic liquid emerging from the ESP), Example 3 (using kerosene-based paraffinic feedstock instead of Fischer-Tropsch based paraffinic feedstock, together with ESP recycle) and Example 4 (using kerosene-based paraffinic feedstock without ESP recycle). These results demonstrate that the combination of Fischer-Tropsch derived paraffinic feedstock together with recycling of the acidic liquid emerging from the ESP back to the liquid linear alkyl benzene sulphonic acid stream leaving the gas/liquid separator is not significantly detrimental to the color of the final linear alkyl benzene sulphonic acid product. Furthermore, the Absorbance of the linear alkyl benzene sulphonic acid produced in Example 1 (using Fischer-Tropsch derived feedstock together with recycle of the acidic liquid emerging from the ESP) is well within the specification guidelines of commercial linear alkyl benzene sulphate products.

It can also be seen from Table 1 that the Direct Acidity, UOM content, water content and sulphuric acid content of the linear alkyl benzene sulphonic acid produced in Example 1 (using Fischer-Tropsch derived paraffinic feedstock together with recycle of the acidic liquid emerging from the ESP) is not significantly different from the Direct Acidity, UOM content, water content and sulphuric acid content of the linear alkyl benzene sulphonic acid produced in Examples 2, 3 and 4.

What is claimed is:

1. A process for preparing an alkyl aryl sulphonic acid comprising the steps of:
(a) contacting an alkyl aromatic hydrocarbon with a gaseous sulphonating agent to produce (i) a first liquid reaction product comprising an alkyl aryl sulphonic acid and (ii) a gaseous effluent stream;
(b) separating the first liquid reaction product from the gaseous effluent stream;
(c) purifying the gaseous effluent stream to provide a cleaned gaseous stream and a second liquid reaction product;
(d) recycling the second liquid reaction product to the first liquid reaction product produced after separation step (b) to produce a third liquid reaction product comprising alkyl aryl sulphonic acid;
wherein the alkyl aromatic hydrocarbon is obtained by contacting an aromatic hydrocarbon with an olefin under alkylating conditions, and wherein said olefin is obtained by dehydrogenation of a Fischer-Tropsch derived paraffinic feedstock.

2. The process of claim 1 wherein the second liquid reaction product comprises an alkyl aryl sulphonic acid and sulphuric acid.

3. The process of claim 1 wherein the gaseous sulphonating agent is sulfur trioxide.

4. The process of claim 1 wherein purification step (c) is carried out by passing the gaseous effluent stream through an electrostatic precipitator.

5. The process of claim 1 wherein sulphonation step (a) is carried out at a temperature in the range of from about 25° C. to about 120° C. and a pressure in the range of from about 30 kPa to about 50 kPa above atmospheric pressure.

6. A process for preparing an alkyl aryl sulphonic acid comprising the steps of:
(a1) contacting an aromatic hydrocarbon with an olefin under alkylating conditions in the presence of an alkylation catalyst to produce an alkyl aromatic hydrocarbon, wherein said olefin is obtained by dehydrogenation of a Fischer-Tropsch derived paraffinic feedstock;
(a) contacting the alkyl aromatic hydrocarbon with a gaseous sulphonating agent to produce (i) a first liquid reaction product (comprising alkyl aryl sulphonic acid) and (ii) a gaseous effluent stream;
(b) separating the first liquid reaction product from the gaseous effluent stream;
(c) purifying the gaseous effluent stream to provide a cleaned gaseous stream and a second liquid reaction product;
(d) recycling the second liquid reaction product to the first liquid reaction product produced after separation step (b) to produce a third liquid reaction product comprising alkyl aryl sulphonic acid.

7. The process of claim 6 wherein the aromatic hydrocarbon is benzene.

8. The process of claim 1 wherein the Fischer-Tropsch derived paraffinic feedstock comprises a mixture of linear and branched paraffins.

9. The process of claim 6 wherein the Fischer-Tropsch derived paraffinic feedstock comprises a mixture of linear and branched paraffins.

10. The process of claim 1 wherein the Fischer-Tropsch derived paraffinic feedstock comprises from 2% to 8% of branched paraffins.

11. The process of claim 6 wherein the Fischer-Tropsch derived paraffinic feedstock comprises from 2% to 8% of branched paraffins.

12. A process for preparing an alkyl aryl sulphonate by neutralizing the alkyl aryl sulphonic acid in the third reaction product prepared to claim 1.

13. A process for preparing an alkyl aryl sulphonate by neutralizing the alkyl aryl sulphonic acid in the third reaction product prepared to claim 6.
